(19) United States
(12) Patent Application Publication
(10) Pub. No.: US 2024/0042136 A1
(43) Pub. Date: Feb. 8, 2024

(54) GLASS SYRINGE BARREL WITH INCREASED FLANGE BREAKING RESISTANCE

(71) Applicant: SCHOTT Pharma Schweiz AG, St. Gallen (CH)

(72) Inventors: Roman Oberhänsli, St. Gallen (CH); Michael Plapp, Amriswil (CH); Raphael Lehner, Teufen (CH)

(73) Assignee: SCHOTT Pharma Schweiz AG, St. Gallen (CH)

(21) Appl. No.: 18/358,985

(22) Filed: Jul. 26, 2023

(30) Foreign Application Priority Data
Jul. 26, 2022 (EP) 22 186 887.0

(51) Int. Cl. A61M 5/31

(52) U.S. Cl. CPC A61M 5/3137; A61M 5/3134

(57) ABSTRACT

A glass syringe barrel is provided that has a bottom end and a top end with a cone region, a shoulder region, a body region, and a flange region therebetween. The glass syringe barrel is configured so that f(x) defines an absolute value of a vertical distance between any point of a first outer contour and a straight line fₒ at a position x, with f(x)=0 at point P₁, wherein x is a horizontal distance between any given point on the straight line fₒ and point P₀ at which the straight line fₒ crosses a line L₁ that runs parallel to the longitudinal axis and that touches an outer surface of the body region, and a maximum value for the term f(x) in a range from x=P₀ to x=P₁ is f(x)ₘₐₓ determined at position xₘₐₓ.

### CROSS REFERENCE TO RELATED APPLICATIONS

This application claims the benefit under 35 USC 119 of European Application 22186887.0 filed Jul. 26, 2022, the entire contents of which are incorporated by reference herein.

### BACKGROUND

### 1. Field of the Invention

The present invention relates to a glass syringe barrel comprising with a top end through which a liquid can be ejected and a bottom end into which a plunger stopper can be pushed, the glass syringe barrel comprising in a direction from the top end to the bottom i) a cone region, ii) a shoulder region, iii) a body region and iv) a flange region, wherein the flange region is characterized by defined shape. The present invention also relates to a plurality of glass syringe barrels, to a process for the preparation of a glass syringe barrel, to a glass syringe barrel obtainable by such a process and to a syringe that comprises a glass syringe barrel.

### 2. Description of Related Art

In order to guarantee a reliable use of pharmaceutical products, pre-filled one-time-use syringes are available commercially. They permit a rapid injection of the product that they contain after a comparatively simple manipulation or handling. This sort of pre-filled syringe has a syringe barrel made from glass or polymer with a syringe head formed on it, in which either a syringe needle is integrated or which has a Luer connecting cone of a conical connection, if necessary, a lockable cone connection (Luer lock). On the side opposite the cone end (i. e., at the open end into which the plunger stopper can be introduced), the syringe body comprises a flange region. By allowing the operator's fingers to be positioned below the flange when the plunger is pushed in, the flange facilitates the injection of a drug located in the syringe when the plunger is pushed in, for example with the operator's thumb.

The aforementioned single-use syringe, also called a ready-made syringe, with a syringe barrel made of glass, is described in Norm DIN ISO 11040, in which, for example, the syringe barrel is described in part 4. The elastomeric standard piston stopper and standard piston rod made of polymer with a cruciform cross section are described in part 5.

A known step in the preparation of pre-filled syringes is the manufacture of a syringe barrel and then the loading of a plurality of those syringe barrels into a so-called nest box, for storage and transport purposes to a location where the syringe barrels will be charged with a medicament and fitted with a plunger or a plug. The nest box comprises a nest tray with a base plate having a plurality of apertures arranged in an array and appropriately sized so that one syringe barrel is closely received in each aperture, with the flange region resting on the upper surface of a base plate. The syringe barrel is loaded into the tray with the lower side of the flange resting on the upper surface of the base plate (as shown in FIG. 1). Usually, the nest box is sealed with a cap and placed in a sterile bag.

However, with syringe bodies known from the prior art it has often been observed that, if they are inserted into the above-described nest box, they often do not rest level but partially canted with the lower side of the flange region resting on the base plate. This “canting” can be countered by forming the lower side of the flange region with as little “indentation” as possible, i.e., with a lower side of the flange region having an outer contour as even as possible. However, such a design of the lower side of the flange region is disadvantageous as it may lead to a reduction of the breaking resistance in the transition area between the flange region and the body region of the syringe body. Such a reduced breaking resistance may result in a breakage of the syringe body in the flange region if the syringes, particularly when being handled in automated filling and sealing machines, hit each other in the flange area.

### SUMMARY

In general, it is an object of the present invention to at least partly overcome a disadvantage arising from the prior art. It is a further object of the present invention to provide a glass syringe barrel comprising a cone region, a shoulder region, a body region and a flange region, which, compared to similar glass syringe barrels known from the prior art, can be inserted into the apertures of a nest box as even as possible (i. e., without “canting”), but which still have a sufficiently high breaking resistance towards forces that are applied onto the flange region of the glass syringe barrel, making the syringe bodies particularly suitable to be handled in automated syringe fill-finish machines. It is a further object of the present invention to provide a glass syringe barrel comprising a cone region, a shoulder region, a body region and a flange region, wherein, compared to similar glass syringe barrels known from the prior art, less force is required to insert a plunger or stopper, also making the syringe barrels particularly suitable to be filled and sealed in automated syringe fill-finish machines. It is a further object of the present invention to provide a glass syringe barrel comprising a cone region, a shoulder region, a body region and a flange region, which, compared to similar glass syringe barrels known from the prior art, can more easily be washed by means of wash liquids that are introduced into the opening of a glass syringe body in an automatically operated cleaning station.

A contribution to at least partly solving at least one, preferably more than one, of the above objects is made by the independent claims. The dependent claims provide preferred embodiments which contribute to at least partly solving at least one of the objects.

A contribution to solving at least one of the objects according to the invention is made by a 1^{st} embodiment of a glass syringe barrel 1 having a longitudinal axis L_{barrel}, the glass syringe barrel comprising a bottom end through which a liquid can be ejected and a top end into which a plunger stopper can be pushed, the glass syringe barrel comprising in a direction from the bottom end to the top end: a cone region having a first end that corresponds to the bottom end of the glass syringe barrel and a second end; a shoulder region having a first end that is adjacent to the second end of the cone region and a second end; a body region with an outer diameter d₁ having a first end that is adjacent to the second end of the shoulder region and a second end; a flange region having at least in parts of the flange region a circular shape, the flange region having a first end that is adjacent to the second end of the body region and a second end that corresponds to the top end of the glass syringe barrel, wherein the flange region has an outer diameter d2>d1 and wherein the flange region is characterized by an outer contour c1 at its first end and an outer contour c2 at its second end; wherein, for any cut surface of the glass syringe barrel that includes the longitudinal axis L_{barrel} and that is obtainable by cutting the glass syringe barrel in a plane at which the shape of the flange region is circular, fₒ and f′ₒ are straight lines that run vertically to longitudinal axis L_{barrel}, wherein fₒ touches the outer contour c1 at its deepest point P₁ and f′ₒ touches the outer contour c2 at its highest point P₂; f(x) defines the absolute value of the vertical distance between any point of the outer contour c1 and straight line fₒ at a position x (with f(x)=0 at point P₁), wherein x is the horizontal distance between any given point on straight line fₒ and point P₀ at which straight line fₒ crosses a line L₁ that runs parallel to longitudinal axis L_{barrel} and that touches the outer surface of the body region and wherein the maximum value for the term f(x) in the range from x=P₀ to x=P₁ is f(x)ₘₐₓ determined at position xₘₐₓ; wherein f′(x) defines the absolute value of the vertical distance between any point of the outer contour c2 and straight line f′ₒ at a position x (with f(x)=0 at point P₂); wherein the following condition is fulfilled: f′(xₘₐₓ)/f(x)ₘₐₓ>1; preferably f′(xₘₐₓ)/f(x)ₘₐₓ>1.2; more preferably f′(xₘₐₓ)/f(x)ₘₐₓ>1.3; even more preferably f′(xₘₐₓ)/f(x)ₘₐₓ>1.4; most preferably f′(xₘₐₓ)/f(x)ₘₐₓ>1.5; wherein it is also preferred that preferably f′(xₘₐₓ)/f(x)ₘₐₓ is less than 5, more preferably less than 3 and most preferably less than 2. Parameter f(x)ₘₐₓ thus represents the deepest indentation on the lower side of the flange region and parameter f′(xₘₐₓ) represents the indention on the upper side of the flange region determined at the same vertical position (i. e., at position x=xₘₐₓ) as the deepest indentation on the lower side of the flange region.

In a preferred embodiment of the glass syringe barrel 1 according to the invention, f′(xₘₐₓ) is in the range from 0.15 to 0.7 mm, preferably in the range from 0.15 to 0.5 mm, more preferably in the range from 0.2 to 0.45 mm and most preferably in the range from 0.2 to 0.4 mm. This preferred embodiment is a 2^{nd} embodiment of glass syringe barrel 1 according to the invention that preferably depends on the 1^{st} embodiment of the invention.

In a further preferred embodiment of the glass syringe barrel 1 according to the invention, f(xₘₐₓ) is in the range from 0.01 to 0.65 mm, preferably in the range from 0.1 to 0.4 mm, more preferably in the range from 0.15 to 0.3 mm and most preferably in the range from 0.2 to 0.3 mm. This preferred embodiment is a 3^{rd} embodiment of glass syringe barrel 1 according to the invention that preferably depends on the 1^{st} or the 2^{nd} embodiment of the invention.

In a further preferred embodiment of the glass syringe barrel 1 according to the invention, for any cut surface of the glass syringe barrel that includes the longitudinal axis L_{barrel} and that is obtainable by cutting the glass syringe barrel in a plane at which the shape of the flange region is circular, D1 is the distance between point P₁ on the outer contour c1 and a point P₃ on outer contour c2, point P₃ being located vertically above point P₁(=thickness of the flange region at its deepest point with f(x)=0) and D2 is the distance between a point of the outer contour c1 and outer contour c2 at position xₘₐₓ, wherein D1 and D2 are both determined in a direction that is parallel to longitudinal axis L_{barrel} and wherein the following condition is fulfilled: D₁/D₂<1.6. preferably D₁/D₂<1.55; more preferably D₁/D₂<1.50; even more preferably D₁/D₂<1.47; most preferably D₁/D₂<1.45; wherein it is also preferred that preferably D₁/D₂ is at least 1.2, more preferably at least 1.3 and most preferably at least 1.4. This preferred embodiment is a 4^{th} embodiment of glass syringe barrel 1 according to the invention that preferably depends on any of the 1^{st} to the 3^{rd} embodiment of the invention.

In a further preferred embodiment of the glass syringe barrel 1 according to the invention, for any cut surface of the glass syringe barrel that includes the longitudinal axis L_{barrel} and that is obtainable by cutting the glass syringe barrel in a plane at which the shape of the flange region is circular, D1 is in the range from 1.85 to 2.5 mm, preferably in the range from 1.88 to 2.4 mm, more preferably in the range from 1.91 to 2.3 mm, even more preferably in the range from 1.94 to 2.2 mm and most preferably in the range from 1.97 to 2.1 mm. This preferred embodiment is a 5^{th} embodiment of glass syringe barrel 1 according to the invention that preferably depends on the 4^{th} embodiment of the invention.

In a further preferred embodiment of the glass syringe barrel 1 according to the invention, for any cut surface of the glass syringe barrel that includes the longitudinal axis L_{barrel} and that is obtainable by cutting the glass syringe barrel in a plane at which the shape of the flange region is circular, D2 is in the range from 1.1 to 2.0 mm, preferably in the range from 1.25 to 1.8 mm, more preferably in the range from 1.30 to 1.6 mm, even more preferably in the range from 1.35 to 1.55 mm and most preferably in the range from 1.4 to 1.5 mm. This preferred embodiment is a 6^{th} embodiment of glass syringe barrel 1 according to the invention that preferably depends on the 4^{th} or the 5^{th} embodiment of the invention.

In a further preferred embodiment of the glass syringe barrel 1 according to the invention, for any cut surface of the glass syringe barrel that includes the longitudinal axis L_{barrel} and that is obtainable by cutting the glass syringe barrel in a plane at which the shape of the flange region is circular, d′ is the distance between point x′ₘₐₓ on the left side of the flange region and point x″ₘₐₓ on the right side of the flange region on straight line fₒ, x′ₘₐₓ and x″ₘₐₓ corresponding to the points at which function f(x) reaches its maximum value on the left side and the right side of the flange region, respectively, wherein d′ is in the range from 12.5 to 15 mm, preferably in the range from 12.5 to 14.5 mm, more preferably in the range from 12.75 to 14 mm and most preferably in the range from 13.0 to 13.5 mm. This preferred embodiment is a 7^{th} embodiment of glass syringe barrel 1 according to the invention that preferably depends on any of the 1^{st} to the 6^{th} embodiment of the invention.

In a further preferred embodiment of the glass syringe barrel 1 according to the invention, for any cut surface of the glass syringe barrel that includes the longitudinal axis L_{barrel} and that is obtainable by cutting the glass syringe barrel in a plane at which the shape of the flange region is circular, d″ is the distance between point P′₁ on the left side of the flange region and point P″₁ on the right side of the flange region on straight line fₒ, wherein d″/d2 is in the range from 0.8 to 0.95, preferably in the range from 0.82 to 0.9, more preferably in the range from 0.83 to 0.87 and most preferably in the range from 0.84 to 0.86. This preferred embodiment is an 8^{th} embodiment of glass syringe barrel 1 according to the invention that preferably depends on any of the 1^{st} to the 7^{th} embodiment of the invention.

In a further preferred embodiment of the glass syringe barrel 1 according to the invention, d″ is in the range from 8 to 20 mm, preferably in the range from 9 to 19 mm, more preferably in the range from 10 to 18.5 mm and most preferably in the range from 11 to 18 mm and d₂ is in the range from 12 to 22 mm, preferably in the range from 14 to 21 mm, more preferably in the range from 16 to 20 mm and most preferably in the range from 17 to 19 mm. This preferred embodiment is a 9^{th} embodiment of glass syringe barrel 1 according to the invention that preferably depends on the 8^{th} embodiment of the invention.

In a further preferred embodiment of the glass syringe barrel 1 according to the invention, for any cut surface of the glass syringe barrel that includes the longitudinal axis L_{barrel} and that is obtainable by cutting the glass syringe barrel in a plane at which the shape of the flange region is circular, a tangent that touches outer contour c2 at position xₘₐₓ includes an angle α with straight line f′ₒ, wherein α is in the range from 10 to 28 degree, preferably in the range from 14 to 26 degree, more preferably in the range from 18 to 25 degree and most preferably in the range from 20 to 24 degree. This preferred embodiment is a 10 embodiment of glass syringe barrel 1 according to the invention that preferably depends on any of the 1^{st} to the 9^{th} embodiment of the invention.

In a further preferred embodiment of the glass syringe barrel 1 according to the invention, for any cut surface of the glass syringe barrel that includes the longitudinal axis L_{barrel} and that is obtainable by cutting the glass syringe barrel in a plane at which the shape of the flange region is circular, L₂ is a straight line at touches the deepest point P′₁ of the outer contour c1 on the left side of the flange region and the deepest point P″₁ of the outer contour c1 on the right side of the flange region; L₃ is a straight line at touches the outer contour c1 on the left side of the flange region at a position x at which f(x) reaches the maximum value f(x)ₘₐₓ and the deepest point P″₁ of the outer contour c1 on the right side of the flange region; wherein straight lines L₂ and L₃ enclose an angle β of less than 3 degree, preferably less than 2.5 degree, more preferably less than 2 degree, more preferably less than 1.5 degree and most preferably less than 1 degree. This preferred embodiment is an 11^{th} embodiment of glass syringe barrel 1 according to the invention that preferably depends on any of the 1^{st} to the 10^{th} embodiment of the invention.

In a further preferred embodiment of the glass syringe barrel 1 according to the invention, d₁ is in the range from 6 to 20 mm, preferably in the range from 7.5 to 16 mm, more preferably in the range from 8 to 14 mm and most preferably in the range from 10 to 12 mm. This preferred embodiment is a 12^{th} embodiment of glass syringe barrel 1 according to the invention that preferably depends on any of the 1^{st} to the 11^{th} embodiment of the invention.

In a further preferred embodiment of the glass syringe barrel 1 according to the invention, difference d₂−d₁ is in the range from 5 to 8 mm, preferably in the range from 6 to 7.7 mm, more preferably in the range from 6.5 to 7.5 mm and most preferably in the range from 6.7 to 7.2 mm. This preferred embodiment is a 13^{th} embodiment of glass syringe barrel 1 according to the invention that preferably depends on any of the 1^{st} to the 12^{th} embodiment of the invention.

In a further preferred embodiment of the glass syringe barrel 1 according to the invention, the wall thickness h in the body region is in the range from 0.6 to 1.6 mm, preferably in the range from 0.7 to 1.5 mm, more preferably in the range from 0.8 to 1.4 mm, even more preferably in the range from 0.9 to 1.3 mm and most preferably in the range from 1 to 1.2 mm. This preferred embodiment is a 14^{th} embodiment of glass syringe barrel 1 according to the invention, that preferably depends on any of the 1^{st} to the 13^{th} embodiment of the invention.

In a further preferred embodiment of the glass syringe barrel 1 according to the invention, the flange region, in a plane that is perpendicular to the longitudinal axis L_{barrel}, has the shape of a circle or the shape of a circle where two opposite sections of the circle have been removed. This preferred embodiment is a 15^{th} embodiment of glass syringe barrel 1 according to the invention, that preferably depends on any of the 1^{st} to the 14^{th} embodiment of the invention.

In a further preferred embodiment of the glass syringe barrel 1 according to the invention, the outer surface of glass syringe barrel in the cone region is roughened or is provided with a coating. This preferred embodiment is a 16^{th} embodiment of glass syringe barrel 1 according to the invention, that preferably depends on any of the 1^{st} to the 15^{th} embodiment of the invention.

In a further preferred embodiment of the glass syringe barrel 1 according to the invention, l₁ is the length of the body region and wherein l₁ is in the range from 30 to 60 mm, preferably in the range from 35 to 55 mm, more preferably in the range from 38 to 50 mm, even more preferably in the range from 39.5 to 45 mm and most preferably in the range from 39.5 to 41 mm. This preferred embodiment is a 17^{th} embodiment of glass syringe barrel 1 according to the invention, that preferably depends on any of the 1^{st} to the 16^{th} embodiment of the invention.

In a further preferred embodiment of the glass syringe barrel 1 according to the invention, the glass syringe barrel has a nominal volume V and wherein V is in a range from 0.5 to 11 ml, preferably from 0.5 to 9 ml, more preferably from 0.5 to 7 ml, even more preferably from 0.5 to 5 ml, most preferably from 0.5 to 3 ml. This preferred embodiment is an 18^{th} embodiment of glass syringe barrel 1 according to the invention, that preferably depends on any of the 1^{st} to the 17^{th} embodiment of the invention.

In a further preferred embodiment of the glass syringe barrel 1 according to the invention, the glass syringe barrel is rotation-symmetric around the longitudinal axis L_{barrel} that runs parallel to the body region and that preferably goes through the center of the top end and the bottom end. This preferred embodiment is a 19^{th} embodiment of glass syringe barrel 1 according to the invention, that preferably depends on any of the 1^{st} to the 18^{th} embodiment of the invention.

In a further preferred embodiment of the glass syringe barrel 1 according to the invention, the glass syringe barrel is designed for a Luer-slip style connector and is sealed with a tip cap that is attached to the at least partially conically shaped upper portion. This preferred embodiment is a 20^{th} embodiment of glass syringe barrel 1 according to the invention, that preferably depends on any of the 1^{st} to the 19^{th} embodiment of the invention.

In a further preferred embodiment of the glass syringe barrel 1 according to the invention, a needle is attached to the at least partially conically shaped upper portion via a Luer connector and wherein the needle is sealed with a needle cap. This preferred embodiment is a 21^{st} embodiment of glass syringe barrel 1 according to the invention, that preferably depends on any of the 1^{st} to the 20^{th} embodiment of the invention.

In a further preferred embodiment of the glass syringe barrel 1 according to the invention, the glass is of a type selected from the group consisting of a borosilicate glass, an aluminosilicate glass, soda lime glass and fused silica. This preferred embodiment is a 22^{nd} embodiment of glass syringe barrel 1 according to the invention, that preferably depends on any of the 1^{st} to the 21^{st} embodiment of the invention.

In a further preferred embodiment of the glass syringe barrel 1 according to the invention, the glass syringe barrel comprises a coating that at least partially superimposes the interior surface of the body region. This preferred embodiment is a 23^{rd} embodiment of glass syringe barrel 1 according to the invention, that preferably depends on any of the 1^{st} to the 22^{nd} embodiment of the invention.

A contribution to solving at least one of the objects according to the invention is made by an embodiment 1 of a process for the production of a glass syringe barrel, more preferably a glass syringe barrel 1 according to the invention, comprising as process steps:
Step I: providing a glass syringe barrel precursor having a longitudinal axis L_{barrel}, the glass syringe barrel precursor comprising a body region with a bottom end and a top end, the body region preferably having an outer diameter d₁;
Step II: heating the top end of the body region, while rotating the glass syringe barrel precursor around its longitudinal axis L_{barrel}, to prepare a ring of molten glass at the top end and forming a flange region by pushing the glass mass out beyond the edge of the body region at the top end of the glass syringe barrel precursor;
Step III: while the glass syringe barrel precursor is rotating around its longitudinal axis L_{barrel}, shaping the flange region obtained in process step II) to obtain a bottom side having an outer contour c1 and a top side having an outer contour c2, wherein the bottom side of the flange region is shaped by means of a shaping tool that comes into contact with the bottom side of the flange region.

To form a flange region by pushing the glass mass out beyond the edge of the body region at the top end of the glass syringe barrel precursor in process step II), different approaches can be applied, which may also depend on the diameter and the thickness of the glass tube and thus also on the size of the glass syringe bodies to be prepared. According to one approach, the mass of molten glass can be pushed out beyond the edge of the body region at the top end of the glass syringe barrel precursor simply by means of the centrifugal force that is applied to the mass of molten glass while the glass syringe barrel precursor is rotating around its longitudinal axis L_{barrel}. According to a further approach, an air stream can be directed onto the mass of molten glass from the top to somehow push the mass of molten glass to the side. In this context it is preferred that this air stream is applied in an angle in a range between 20 to 70 degrees, preferably in the range from 40 to 60 degrees, relative to longitudinal axis L_{barrel}. Also, this air stream is preferably applied in an amount of 8 to 17 NL/min, more preferably in an amount of 10 to 14 NL/min.

In process step III) a moulding tool is moved directly to the bottom side of the flange region, preferably by means of a lifting cylinder. By means of this molding tool it is prevented that the flange region formed in process step II), which is still in a molten state, deforms downwards. The moulding tool thus stabilises the flange and ensures the flange height position. In this context it is preferred that the whole process of forming the final shape of the flange region does not require the use of any oil to actively lubricate the machine tools used to form the final shape of the flange region.

In a preferred embodiment of process 1 according to the invention in process step III) the flange region is brought into the desired flange shape by additionally directing an air stream to the top side of the flange region. The blown-on glass surface cools down faster and a sagging of the flange is thus prevented. In this context it is preferred that this air stream is applied in an angle in a range between 40 to 80 degrees, preferably in the range between 50 to 70 degrees, relative to longitudinal axis L_{barrel}. Also, the air stream is preferably applied in an amount of 4 to 10 NL/min, more preferably in an amount of 6 to 8 NL/min. This preferred embodiment is a 2^{nd} embodiment of process 1 according to the invention, that preferably depends on the 1^{st} embodiment of the invention.

In a further preferred embodiment of process 1 according to the invention, the shaping tool used to shape the bottom side of the flange region is made of carbon, preferably graphite R7710. This preferred embodiment is a 3^{rd} embodiment of process 1 according to the invention, that preferably depends on the 1^{st} or the 2^{nd} embodiment of the invention.

In a further preferred embodiment of process 1 according to the invention, in process steps II) and III) the glass syringe barrel precursor is rotating around its longitudinal axis L_{barrel} with a rotating speed in the range from 700 to 900 rpm, preferably in the range from 740 to 790 rpm. This preferred embodiment is a 4^{th} embodiment of process 1 according to the invention, that preferably depends on any of the 1^{st} to the 3^{rd} embodiment of the invention.

In a further preferred embodiment of process 1 according to the invention, shaping in process step III) is performed under such conditions that a flange region is obtained having at least in parts of the flange region a circular shape, the flange region having a first end that is adjacent to the second end of the body region and a second end that corresponds to the top end of the glass syringe barrel, wherein the flange region has an outer diameter d₂>d₁ and wherein the flange region is characterized by an outer contour c1 at its first end and an outer contour c2 at its second end. This preferred embodiment is a 5^{th} embodiment of process 1 according to the invention, that preferably depends on any of the 1^{st} to the 4^{th} embodiment of the invention.

In a further preferred embodiment of process 1 according to the invention, shaping in process step III) is performed under such conditions that a flange region is obtained in which, for any cut surface of the glass syringe barrel that includes the longitudinal axis L_{barrel} and that is obtainable by cutting the glass syringe barrel in a plane at which the shape of the flange region is circular, fₒ and f′ₒ are straight lines that run vertically to longitudinal axis L_{barrel}, wherein fₒ touches the outer contour c1 at its deepest point P₁ and f′ₒ touches the outer contour c2 at its highest point P₂; f(x) defines the absolute value of the vertical distance between any point of the outer contour c1 and straight line fₒ at a position x (with f(x)=0 at point P₁), wherein x is the horizontal distance between any given point on straight line fₒ and point P₀ at which straight line fₒ crosses a line L₁ that runs parallel to longitudinal axis L_{barrel} and that touches the outer surface of the body region and wherein the maximum value for the term f(x) in the range from x=P₀ to x=P₁ is f(x)ₘₐₓ determined at position xₘₐₓ; wherein f′(x) defines the absolute value of the vertical distance between any point of the outer contour c2 and straight line f′ₒ at a position x (with f(x)=0 at point P₂); wherein the following condition is fulfilled: f′(xₘₐₓ)/f(x)ₘₐₓ>1; preferably f′(xₘₐₓ)/f(x)ₘₐₓ>1.2; more preferably f′(xₘₐₓ)/f(x)ₘₐₓ>1.3; even more preferably f′(xₘₐₓ)/f(x)ₘₐₓ>1.4; most preferably f′(xₘₐₓ)/f(x)ₘₐₓ>1.5; wherein it is also preferred that preferably f′(xₘₐₓ)/f(x)ₘₐₓ is less than 5, more preferably less than 3 and most preferably less than 2.

This preferred embodiment is a 6^{th} embodiment of process 1 according to the invention, that preferably depends on any of the 1^{st} to the 5^{th} embodiment of the invention.

In a further preferred embodiment of process 1 according to the invention, shaping in process step III) is performed under such conditions that a flange region is obtained in which f′(xₘₐₓ) is in the range from 0.15 to 0.7 mm, preferably in the range from 0.15 to 0.5 mm, more preferably in the range from 0.2 to 0.45 mm and most preferably in the range from 0.2 to 0.4 mm. This preferred embodiment is an 7^{th} embodiment of process 1 according to the invention that preferably depends on the 6^{th} embodiment of the invention.

In a further preferred embodiment of process 1 according to the invention, shaping in process step III) is performed under such conditions that a flange region is obtained in which f(xₘₐₓ) is in the range from 0.01 to 0.65 mm, preferably in the range from 0.1 to 0.4 mm, more preferably in the range from 0.15 to 0.3 mm and most preferably in the range from 0.2 to 0.3 mm. This preferred embodiment is an 8^{th} embodiment of process 1 according to the invention that preferably depends on the 6^{th} or the 7^{th} embodiment of the invention.

In a further preferred embodiment of process 1 according to the invention, shaping in process step III) is performed under such conditions that a flange region is obtained in which, for any cut surface of the glass syringe barrel that includes the longitudinal axis L_{barrel} and that is obtainable by cutting the glass syringe barrel in a plane at which the shape of the flange region is circular, D1 is the distance between point P₁ on the outer contour c1 and a point P₃ on outer contour c2, point P₃ being located vertically above point P₁ (=thickness of the flange region at its deepest point) and D2 is the distance between a point of the outer contour c1 and outer contour c2 at position xₘₐₓ, wherein D1 and D2 are both determined in a direction that is parallel to longitudinal axis L_{barrel} and wherein the following condition is fulfilled: D₁/D₂<1.6. preferably D₁/D₂<1.55; more preferably D₁/D₂<1.50; even more preferably D₁/D₂<1.47; most preferably D₁/D₂<1.45; wherein it is also preferred that preferably D₁/D₂ is at least 1.2, more preferably at least 1.3 and most preferably at least 1.4. This preferred embodiment is a 9^{th} embodiment of process 1 according to the invention that preferably depends on any of the 6^{th} to the 8^{th} embodiment of the invention.

In a further preferred embodiment of process 1 according to the invention, shaping in process step III) is performed under such conditions that D1 is in the range from 1.85 to 2.5 mm, preferably in the range from 1.88 to 2.4 mm, more preferably in the range from 1.91 to 2.3 mm, even more preferably in the range from 1.94 to 2.2 mm and most preferably in the range from 1.97 to 2.1 mm. This preferred embodiment is a 10^{th} embodiment of process 1 according to the invention that preferably depends on the 9^{th} embodiment of the invention.

In a further preferred embodiment of process 1 according to the invention, shaping in process step III) is performed under such conditions that D2 is in the range from 1.1 to 2.0 mm, preferably in the range from 1.25 to 1.8 mm, more preferably in the range from 1.30 to 1.6 mm, even more preferably in the range from 1.35 to 1.55 mm and most preferably in the range from 1.4 to 1.5 mm. This preferred embodiment is an 11^{th} embodiment of process 1 according to the invention that preferably depends on the 9^{th} or the 10^{th} embodiment of the invention.

In a further preferred embodiment of process 1 according to the invention, shaping in process step III) is performed under such conditions that a flange region is obtained in which, for any cut surface of the glass syringe barrel that includes the longitudinal axis L_{barrel} and that is obtainable by cutting the glass syringe barrel in a plane at which the shape of the flange region is circular, d′ is the distance between point x′ₘₐₓ on the left side of the flange region and point x″ₘₐₓ on the right side of the flange region on straight line fₒ, x′ₘₐₓ and x″ₘₐₓ corresponding to the points at which function f(x) reaches its maximum value on the left side and the right side of the flange region, respectively, wherein d′ is in the range from 12.5 to 15 mm, preferably in the range from 12.5 to 14.5 mm, more preferably in the range from 12.75 to 14 mm and most preferably in the range from 13.0 to 13.5 mm. This preferred embodiment is a 12^{th} embodiment of process 1 according to the invention that preferably depends on any of the 6^{th} to the 11^{th} embodiment of the invention.

In a further preferred embodiment of process 1 according to the invention, shaping in process step III) is performed under such conditions that a flange region is obtained in which, for any cut surface of the glass syringe barrel that includes the longitudinal axis L_{barrel} and that is obtainable by cutting the glass syringe barrel in a plane at which the shape of the flange region is circular, d″ is the distance between point P′₁ on the left side of the flange region and point P″1 on the right side of the flange region on straight line fₒ, wherein d″/d2 is in the range from 0.8 to 0.95, preferably in the range from 0.82 to 0.9, more preferably in the range from 0.83 to 0.87 and most preferably in the range from 0.84 to 0.86. This preferred embodiment is a 13^{th} embodiment of process 1 according to the invention that preferably depends on any of the 6^{th} to the 12^{th} embodiment of the invention.

In a further preferred embodiment of process 1 according to the invention, shaping in process step III) is performed under such conditions that a flange region is obtained in which d″ is in the range from 8 to 20 mm, preferably in the range from 9 to 19 mm, more preferably in the range from 10 to 18.5 mm and most preferably in the range from 11 to 18 mm and d₂ is in the range from 12 to 22 mm, preferably in the range from 14 to 21 mm, more preferably in the range from 16 to 20 mm and most preferably in the range from 17 to 19 mm. This preferred embodiment is a 14^{th} embodiment of process 1 according to the invention that preferably depends on the 13^{th} embodiment of the invention.

In a further preferred embodiment of process 1 according to the invention, shaping in process step III) is performed under such conditions that a flange region is obtained in which, for any cut surface of the glass syringe barrel that includes the longitudinal axis L_{barrel} and that is obtainable by cutting the glass syringe barrel in a plane at which the shape of the flange region is circular, a tangent that touches outer contour c2 at position xₘₐₓ includes an angle α with straight line f′ₒ, wherein α is in the range from 14 to 26 degree, more preferably in the range from 18 to 25 degree and most preferably in the range from 20 to 24 degree. This preferred embodiment is a 15^{th} embodiment of process 1 according to the invention that preferably depends on any of the 6^{th} to the 14^{th} embodiment of the invention.

In a further preferred embodiment of process 1 according to the invention, shaping in process step III) is performed under such conditions that a flange region is obtained in which, for any cut surface of the glass syringe barrel that includes the longitudinal axis L_{barrel} and that is obtainable by cutting the glass syringe barrel in a plane at which the shape of the flange region is circular, L₂ is a straight line at touches the deepest point P′₁ of the outer contour c1 on the left side of the flange region and the deepest point P″₁ of the outer contour c1 on the right side of the flange region; L₃ is a straight line at touches the outer contour c1 on the left side of the flange region at a position x at which f(x) reaches the maximum value f(x)ₘₐₓ and the deepest point P″₁ of the outer contour c1 on the right side of the flange region; wherein straight lines L₂ and L₃ enclose an angle β of less than 3 degree, preferably less than 2.5 degree, more preferably less than 2 degree, more preferably less than 1.5 degree and most preferably less than 1 degree. This preferred embodiment is a 16^{th} embodiment of process 1 according to the invention that preferably depends on any of the 6^{th} to the 15^{th} embodiment of the invention.

In a further preferred embodiment of process 1 according to the invention, shaping in process step III) is performed under such conditions that a flange region with d₁ is in the range from 6 to 20 mm, preferably in the range from 7.5 to 16 mm, more preferably in the range from 8 to 14 mm and most preferably in the range from 10 to 12 mm. This preferred embodiment is a 17^{th} embodiment of process 1 according to the invention that preferably depends on any of the 6^{th} to the 16^{th} embodiment of the invention.

In a further preferred embodiment of process 1 according to the invention, shaping in process step III) is performed under such conditions that a flange region is obtained in which d₂-d₁ is in the range from 5 to 8 mm, preferably in the range from 6 to 7.7 mm, more preferably in the range from 6.5 to 7.5 mm and most preferably in the range from 6.7 to 7.2 mm. This preferred embodiment is an 18^{th} embodiment of process 1 according to the invention that preferably depends on any of the 6^{th} to the 17^{th} embodiment of the invention.

In a further preferred embodiment of process 1 according to the invention, shaping in process step III) is performed by adjusting at least one of the following parameters:
(a) the speed at which the glass syringe barrel precursor rotates around its longitudinal axis L_{barrel};
(b) the temperature of the glass in the ring of molten glass;
(c) the pressure with which the gas stream that is directed onto the top of the ring of molten glass;
(d) the position at which the shaping tool is located beneath the ring of molten glass.

Particularly preferred embodiments of process 1 according to the present invention are characterized in that the following parameters or combination of parameters are adjusted: a), b), c), d), a)b), a)c), a)d), b)c), b)d), c)d), a)b)c), a)b)d), b)c)d) and a)b)c)d), wherein the combination a)b)c)d) is most preferred. This preferred embodiment is a 19^{th} embodiment of process 1 according to the invention, that preferably depends on any of the 1^{st} to the 18^{th} embodiment of the invention.

In a further preferred embodiment of process 1 according to the present invention, the thickness h of the glass in the body region of the glass syringe barrel precursor is in the range from 0.6 to 1.6 mm, preferably in the range from 0.7 to 1.5 mm, more preferably in the range from 0.8 to 1.4 mm, even more preferably in the range from 0.9 to 1.3 mm and most preferably in the range from 1 to 1.2 mm. This preferred embodiment is a 20^{th} embodiment of process 1 according to the invention, that preferably depends on any of the 1^{st} to the 19^{th} embodiment of the invention.

In a further preferred embodiment of process 1 according to the present invention, the glass syringe barrel precursor provided in process step I) further comprises: a cone region having a first end that corresponds to the bottom end of the glass syringe barrel precursor and a second end; a shoulder region having a first end that is adjacent to the second end of the cone region and a second end that corresponds to the bottom end of the body region.

This preferred embodiment is a 21^{st} embodiment of process 1 according to the invention, that preferably depends on any of the 1^{st} to the 20^{th} embodiment of the invention.

In a further preferred embodiment of process 1 according to the present invention, the process further comprises the step of: roughening the outer surface of glass syringe barrel in the cone region or applying coating onto the outer surface of glass syringe barrel in the cone region.

This preferred embodiment is a 22^{nd} embodiment of process 1 according to the invention, that preferably depends on any of the 1^{st} to the 21^{st} embodiment of the invention.

In a further preferred embodiment of process 1 according to the present invention, the process further comprises the step of: cutting off two parts of the flange region that are located on opposing sides of the flange region, wherein cutting can be accomplished mechanically by means of a cutting device or can be accomplished thermally by means of a flame with which the glass is molten at the predetermined position and is then pulled apart.

This preferred embodiment is a 23^{rd} embodiment of process 1 according to the invention, that preferably depends on any of the 1^{st} to the 22^{nd} embodiment of the invention.

In a further preferred embodiment of process 1 according to the present invention, the glass is of a type selected from the group consisting of a borosilicate glass, an aluminosilicate glass and fused silica. This preferred embodiment is a 24^{th} embodiment of process 1 according to the invention, that preferably depends on any of the 1^{st} to the 23^{rd} embodiment of the invention.

In a further preferred embodiment of process 1 according to the present invention, the process further comprises the step of: superimposing at least a part of the interior surface of the body region with a coating.

This preferred embodiment is a 25^{th} embodiment of process 1 according to the invention, that preferably depends on any of the 1^{st} to the 24^{th} embodiment of the invention.

A contribution to solving at least one of the objects according to the invention is made by an embodiment 1 of a glass syringe barrel 2 obtainable by the process of the invention according to the present invention, preferably by the process 1 according to any of its preferred embodiments. In a preferred embodiment of the glass syringe barrel 2, this glass syringe barrel 2 shows the technical features of the glass syringe barrel 1 of the invention.

A contribution to solving at least one of the objects according to the invention is made by a plurality of glass syringe barrels 1 or 2 according to the present invention, preferably a plurality of glass syringe barrels 1 according to any of its preferred embodiments, wherein at least one of the following conditions, preferably both of them, is/are fulfilled:
the relative standard deviation of the flange thickness D1 is less than 6%, preferably less than 5.5% and more preferably less than 5%;
the relative standard deviation of the length l₂ of the syringe is less than 12%, preferably less than 11.5% and more preferably less than 11%.

According to a preferred embodiment of the plurality of glass syringe barrels 1 or 2 according to the present invention, at least one of the following conditions, preferably both of them, is/are fulfilled:
the relative standard deviation of the flange thickness D1
in case of a syringe having an outside diameter d₁ in the range from 7.5 to 8.5 mm, preferably in the range from 8 to 8.2 mm and a length l₂ in the range from 60 to 70 mm, preferably in the range from 65 to 67 mm, is less than 3%, preferably less than 2.75% and more preferably less than 2.5%;
in case of a syringe having an outside diameter d₁ in the range from 10 to 12 mm, preferably in the range from 10.5 to 11 mm and a length l₂ in the range from 45 to 55 mm, preferably in the range from 47 to 50 mm, is less than 3%, preferably less than 2.75% and more preferably less than 2.5%;
in case of a syringe having an outside diameter d₁ in the range from 10 to 12 mm, preferably in the range from 10.5 to 11 mm and a length l₂ in the range from 45 to 60 mm, preferably in the range from 52 to 54 mm, is less than 2.0%, preferably less than 1.75% and more preferably less than 1.5%;
in case of a syringe having an outside diameter d₁ in the range from 10 to 12 mm, preferably in the range from 10.5 to 11 mm and a length l₂ in the range from 50 to 60 mm, preferably in the range from 54 to 56 mm, is less than 2.5%, preferably less than 2.25% and more preferably less than 2.0%;
in case of a syringe having an outside diameter d₁ in the range from 10 to 12 mm, preferably in the range from 10.5 to 11 mm and a length l₂ in the range from 60 to 75 mm, preferably in the range from 66 to 68 mm, is less than 5%, preferably less than 4.75% and more preferably less than 4.5%;
in case of a syringe having an outside diameter d₁ in the range from 10 to 12 mm, preferably in the range from 10.5 to 11 mm and a length l₂ in the range from 75 to 95 mm, preferably in the range from 84 to 86 mm, is less than 3%, preferably less than 2.75% and more preferably less than 2.5%;
the relative standard deviation of the length l₂ of the syringe
in case of a syringe having an outside diameter d₁ in the range from 7.5 to 8.5 mm, preferably in the range from 8 to 8.2 mm and a length l₂ in the range from 60 to 70 mm, preferably in the range from 65 to 67 mm, is less than 10%, preferably less than 9.5% and more preferably less than 9%;
in case of a syringe having an outside diameter d₁ in the range from 10 to 12 mm, preferably in the range from 10.5 to 11 mm and a length l₂ in the range from 45 to 55 mm, preferably in the range from 47 to 50 mm, is less than 11.0%, preferably less than 10.5% and more preferably less than 10%;
in case of a syringe having an outside diameter d₁ in the range from 10 to 12 mm, preferably in the range from 10.5 to 11 mm and a length l₂ in the range from 45 to 60 mm, preferably in the range from 52 to 54 mm, is less than 10.5%, preferably less than 10% and more preferably less than 9.5%;
in case of a syringe having an outside diameter d₁ in the range from 10 to 12 mm, preferably in the range from 10.5 to 11 mm and a length l₂ in the range from 50 to 60 mm, preferably in the range from 54 to 56 mm, is less than 9.5%, preferably less than 9% and more preferably less than 8.5%;
in case of a syringe having an outside diameter d₁ in the range from 10 to 12 mm, preferably in the range from 10.5 to 11 mm and a length l₂ in the range from 60 to 75 mm, preferably in the range from 66 to 68 mm, is less than 9.5%, preferably less than 9% and more preferably less than 8.5%;
in case of a syringe having an outside diameter d₁ in the range from 10 to 12 mm, preferably in the range from 10.5 to 11 mm and a length l₂ in the range from 75 to 95 mm, preferably in the range from 84 to 86 mm, is less than 11%, preferably less than 10.5% and more preferably less than 10%; wherein 12 is the length of the upper most end on top of the flange region determined at position P₂ to the bottom end (i. e. the lower end of the conically shaped region).

The relative standard deviation of the of the maximum flange thickness D1 and of length l₂ of the syringe in % is the standard deviation relative to the average value determined for these parameters in the plurality of syringe bodies.

“A plurality of glass syringes” in the sense of the present invention preferably comprises at least 10 glass syringes, preferably at least 25 glass syringes, more preferably at least 50 glass syringes, even more preferably at least 100 glass syringes even more preferably at least 200 glass syringes and most preferably at least 400 glass syringes. Furthermore, the plurality of glass syringes preferably has been collected arbitrarily and particularly has not been selected with regard to any property. For example, the plurality glass syringes may be the group of syringes which are packed together in a typical transport tray.

A contribution to solving at least one of the objects according to the invention is made by an embodiment 1 of a syringe comprising: a glass syringe barrel 1 according to the present invention, preferably according to any of its preferred embodiments, a plurality 1 of glass syringe barrels according to the present invention, preferably according to any of its preferred embodiments, or the glass syringe barrel 2; a plunger that has been pushed into the bottom end of the glass syringe barrel or into the bottom end of each of the glass syringe barrels contained in the plurality 1 of glass syringe barrels.

In a further preferred embodiment of the syringe according to the present invention, the syringe further comprises: a pharmaceutical composition that is filled into at least a part of the inner volume of the body region.

### Glass Syringe Barrel

The glass syringe barrel according to the invention may have any size or shape which the skilled person deems appropriate in the context of the invention. The bottom end of the glass syringe barrel comprises an opening in the form of a channel located within the cone region through which a pharmaceutical composition that is contained in the glass syringe can be squeezed out of the glass syringe barrel and a top end with a flange region into which a plunger stopper can be pushed. Preferably, the glass syringe barrel is of a one-piece design that is prepared by providing a glass tube, preferably in form of a hollow cylinder, and forming the desired shape of the flange region. A preferred glass syringe barrel is a prefilled glass syringe barrel that is filled with a pharmaceutical preparation. Preferably, the glass syringe barrel is rotationally symmetrical around the longitudinal axis L_{barrel} that preferably goes perpendicular through the centre of the body region.

### Glass

The glass of the glass syringe barrel be any type of glass and may consist of any material or combination of materials which the skilled person deems suitable in the context of the invention. Preferably, the glass is suitable for pharmaceutical packaging. Particularly preferable, the glass is of type I, more preferably type I b, in accordance with the definitions of glass types in section 3.2.1 of the European Pharmacopoeia, 7^{th} edition from 2011. Additionally, or alternatively preferable to the preceding, the glass is selected from the group consisting of a borosilicate glass, an aluminosilicate glass, soda lime glass and fused silica; or a combination of at least two thereof. For the use in this document, an aluminosilicate glass is a glass which has a content of Al₂O₃ of more than 8 wt.-%, preferably more than 9 wt.-%, particularly preferable in a range from 9 to 20 wt.-%, in each case based on the total weight of the glass. A preferred aluminosilicate glass has a content of B₂O₃ of less than 8 wt.-%, preferably at maximum 7 wt.-%, particularly preferably in a range from 0 to 7 wt.-%, in each case based on the total weight of the glass. For the use in this document, a borosilicate glass is a glass which has a content of B₂O₃ of at least 1 wt.-%, preferably at least 2 wt.-%, more preferably at least 3 wt.-%, more preferably at least 4 wt.-%, even more preferably at least 5 wt.-%, particularly preferable in a range from 5 to 15 wt.-%, in each case based on the total weight of the glass. A preferred borosilicate glass has a content of Al₂O₃ of less than 7.5 wt.-%, preferably less than 6.5 wt.-%, particularly preferably in a range from 0 to 5.5 wt.-%, in each case based on the total weight of the glass. In a further aspect, the borosilicate glass has a content of Al₂O₃ in a range from 3 to 7.5 wt.-%, preferably in a range from 4 to 6 wt.-%, in each case based on the total weight of the glass.

A glass which is further preferred according to the invention is essentially free from B. Therein, the wording “essentially free from B” refers to glasses which are free from B which has been added to the glass composition by purpose. This means that B may still be present as an impurity, but preferably at a proportion of not more than 0.1 wt.-%, more preferably not more than 0.05 wt.-%, in each case based on the weight of the glass.

### Shape of the Flange Region

An important element of the glass syringe barrel 1 according to the invention is the shape of the flange region that is formed in process step III) of the process 1 according to the present invention, particularly the outer contour c1 at the first end of the flange region and the outer contour c2 at the second end of the flange region. The outer contour c1 and the outer contour c2 are characterized in that ratio of the depth of indention on the second end to the depth of indention on the first end (f′(xₘₐₓ)/f(x)ₘₐₓ) as determined at a position at which the indention on the end first end riches its maximum value is >1. Surprisingly, it has been discovered that, if the outer contour c1 at the first end of the flange region and the outer contour c2 at the second end of the flange region are characterized by such a relative depth of indention, not only the positioning of the glass syringe barrels in the nest can be significantly improved by reducing the risk of the syringes “canting” and thus not coming to rest straight with the bottom side of the flange region on the base plate, the flange breaking resistance can also be significantly improved.

### Pharmaceutical Composition

In the context of the invention, every liquid pharmaceutical composition which the skilled person deems suitable to be used in a syringe comes into consideration. A pharmaceutical composition is a composition comprising at least one active ingredient. A preferred active ingredient is a vaccine. A further preferred pharmaceutical composition is a parenterialium, i.e. a composition which is intended to be administered via the parenteral route, which may be any route which is not enteral. Parenteral administration can be performed by injection, e.g. using a needle (usually a hypodermic needle) and a syringe.

### Measurement Methods

The following measurement methods are to be used in the context of the invention. Unless otherwise specified, the measurements have to be carried out at an ambient temperature of 23° C., an ambient air pressure of 100 kPa (0.986 atm) and a relative atmospheric humidity of 50%.

Determination of f(x) and f′(x)

The outer contour c1 and c2 of the glass syringe barrel in the flange region defined by the function f(x) and f′(x), respectively, can be determined in a non-destructive manner using a profile projector. This approach is particularly suitable for glass syringe barrels that have been chemically and/or thermally tempered and that therefore cannot be easily sliced in half without the glass cracking or bursting. For determining the values for f(x) and f′(x) in a non-destructive manner the outer contour c1 and c2 of the glass syringe barrel in the flange region is visualized using a Mitutoyo PJ-3000 profile projector. The profile projector has a 10× magnification and is operated with transmitted light illumination. The barrels are placed in Hallbrite® BHB (a butyloctyl salicylate obtainable from the Hallstar Company, Chicago, USA), which is filled into a glass bowl. Hallbrite® BHB is used to visualize the outer contour of the flange region. It is ensured that the cross-section of the glass syringe barrel that is inspected in the profile projector corresponds to the plane that is centrically located in the glass syringe barrel and that comprises the longitudinal axis L_{barrel} of the glass syringe barrel, i. e. the axis that goes perpendicular through the barrel (see FIGS. 6A and 6B).

To improve the measuring accuracy, the outer contour c1 and c2 of the glass syringe barrel in the flange region can also be determined from a physical cross-sectional cut parallel along to the longitudinal axis L_{barrel} of the barrel (it is again ensured that the cross-section of the glass syringe barrel corresponds to the plane that is centrically located in the glass syringe barrel and that comprises the longitudinal axis of the glass syringe barrel as shown in FIGS. 6A and 6B). For preparation without breakage, the barrel may be embedded into transparent 2-component epoxy resin, for example STRUERS GmbH, EpoFix Resin, or other suitable materials. After curing of the epoxy resin, a cross-sectional cut parallel along to the barrel axis can be achieved by machine-supported sawing, grinding and polishing. Geometrical features of the barrel can then be determined (measured) by means of non-distorting image capturing and geometrical analysis software tools.

The relevant outer contour c1 and c2 of the glass syringe barrel in the flange region can be extracted and numerically approximated from the images obtained by means of the two approaches described above. For the extraction of the relevant contour, the images undergo the image processing steps implemented in Python [https://www.python.org/] based on the image processing library OpenCV [https://open-cv.org/].

First, the images are denoised using a median filter. The denoised images are then processed with an edge detection algorithm based on a Sobel filter, in which the contours are identified by thresholding the gradient image.

### Determination of the Flange Thickness D1

The flange thickness D1 is determined by means of a Round Flange Camera.

### EXAMPLE

A glass tube (FIOLAX® clear, SCHOTT AG, Germany) having an outer diameter d₁ of 10.85 mm and a wall thickness n of 1.1 mm made of borosilicate glass is loaded into the head of a rotary machine. While rotating around its longitudinal axis L_{tube} one end of the glass tube (i. e. at the end of which the Luer cone will be localized) is heated to its softening point with flames (see FIG. 11A). While the glass tube is rotating around its longitudinal axis the end that has been heated is shaped using molding tools that act on predetermined positions of the outer surface of the glass tube at the first end to form the cone region. In a further process step the glass tube, while rotating around its longitudinal, is cut at a predetermined position above the first end to obtain a glass tube comprising a first end that is cone shaped and second end. The glass tube of the thus obtained glass syringe barrel precursor is then heated at the second end, while rotating around its longitudinal, to a temperature above its glass transition temperature with a flame. While the glass tube is still rotating around its longitudinal at a rotational speed of 750 rpm, the mass of molten glass is pushed out beyond the edge of the body region at the top end of the glass syringe barrel precursor simply by means of the centrifugal force, thereby forming a flange region.

In a further process step, the final shape of the flange region is obtained by means of the set up shown in FIG. 9. As shown in that figure, the final shape on the bottom side of the flange region is prepared by bringing the mass of molten glass into contact with a shaping tool in such a way that the shaping tool somehow lifts the mass of glass upwards, thereby reducing the deepest indentation on the lower side of the flange region (f(x)ₘₐₓ). At the same time, the top side of the flange region is cooled by means of an air stream (applied in an angle of 50°) that primarily serves to cool down the surface on the top side of the flange region, thereby stabilizing the shape of the flange region on that side. In a Comparative Example, process step III) (i. e., the treatment with the shaping tool and the air stream) has been omitted.

| | Comparative Example 1 | Example 1 |
|---|---|---|
| f′ (xₘₐₓ)/f(x)ₘₐₓ | 0.08 | 1.33 |
| f′ (x)ₘₐₓ [mm] | 0.06 | 0.32 |
| f(x)ₘₐₓ [mm] | 0.77 | 0.24 |
| α [degree] | 8 | 22 |
| β [degree] | 3.17 | 0.97 |
| D₁ [mm] | 1.81 | 1.99 |
| D₂ [mm] | 1.05 | 1.42 |
| d′ [mm] | 12.29 | 13.27 |
| d″ [mm] | 15.67 | 15.23 |
| d₂ [mm] | 17.55 | 17.88 |

It has been observed that the glass syringe barrels according to the present invention do rest more level when being placed in a nest box as (i. e., they are less prone to “canting”), compared to the glass syringe barrels known from the prior art.

### BRIEF DESCRIPTION OF THE DRAWINGS

FIG. 1 shows a cross-sectional view of a nest 200 holding glass syringe barrels 100;

FIG. 2 shows a cross-sectional enlarged view a syringe barrel 100 according to the present invention;

FIG. 3 shows a cross-sectional enlarged view of a part of the flange region 113 in a glass syringe barrel 100 according to the present invention and the determination of the outer contour c1 and c2 by means of functions f(x) and f′(x);

FIG. 4 shows in a further a cross-sectional enlarged view of the shape of a flange region 113 in a glass syringe barrel 100 according to the present invention and the determination of P;

FIG. 5 shows in a further a cross-sectional enlarged view of the shape of a flange region 113 in a glass syringe barrel 100 according to the present invention and the determination of d′ and d″;

FIG. 6A shows in a side view the localization of plane 116 that is used to determine the outer contour c1 and c2 in the flange region 113;

FIG. 6B shows in a top view the localization of plane 116 that is used to determine the outer contour c1 and c2 in the flange region 113;

FIGS. 7A-7B show a top-view two different shapes of the flange region;

FIG. 8 shows a cross-sectional enlarged view of a part a syringe according to the present invention into which a plunger 118 has been inserted into the opening at the side of the flange region 113;

FIG. 9 illustrates step III) of process 1 according to the invention for the preparation of a glass syringe barrel 100.

### DETAILED DESCRIPTION

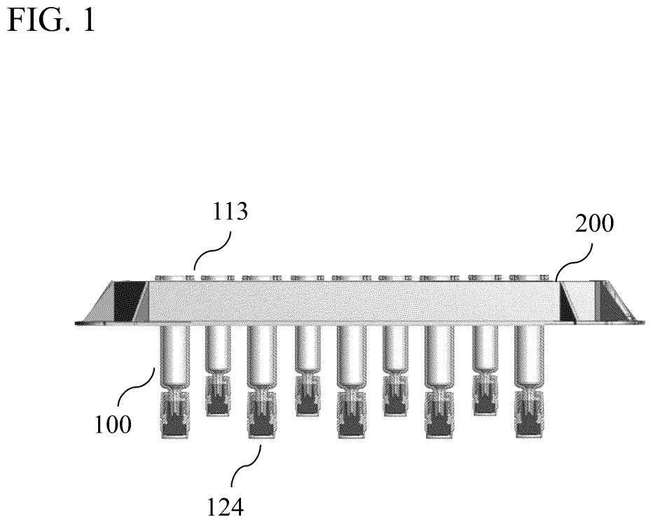

FIG. 1 shows a cross-sectional view of a nest 200 that is holding a plurality of glass syringe barrels 100. Such a nest 200 is used in automated syringe fill-finish machines, in which a plurality of syringe barrels 100 that are closed at the conus-shaped end by means of appropriate closure systems 124 are filled with a pharmaceutical composition. The nest 200 comprises a plurality of recesses (not visible in FIG. 1) into which the syringe barrels 100 are introduced in such a way that they rest with the lower (first) end 114 on the upper surface of the nest 200.

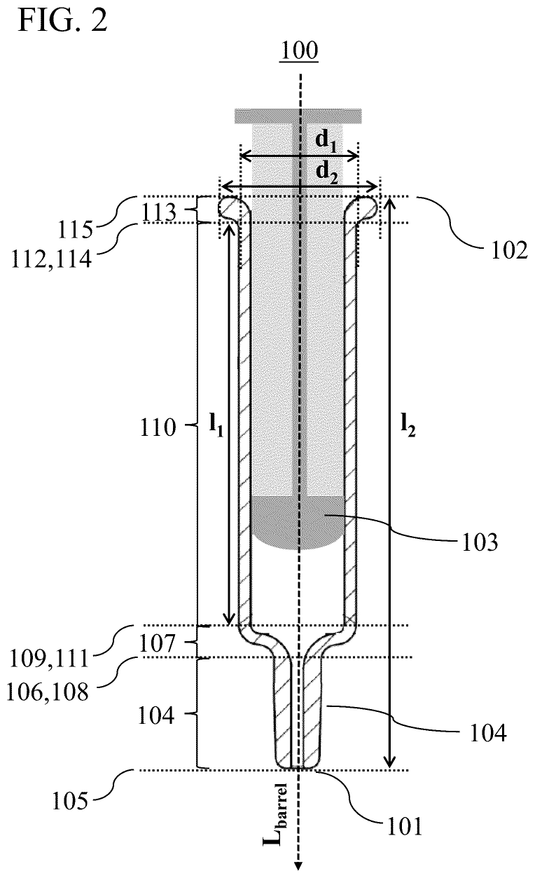

FIG. 2 shows a cross-sectional enlarged view a syringe barrel 100 according to the present invention. The syringe barrel 100 has a longitudinal axis L_{barrel} and comprises a bottom end 101 through which a liquid can be ejected and which is usually shaped in the form of a conus, and a top end 102 into which a plunger stopper 103 (or a plug) can be pushed. The glass syringe barrel 100 comprises in a direction from the bottom end 101 to the top end 102 a cone region 104 having a first end 105 that corresponds to the bottom end 101 of the glass syringe barrel 100 and a second end 106, a shoulder region 107 having a first end 108 that is adjacent to the second end 106 of the cone region 104 and a second end 109, a body region 110 with an outer diameter d₁ having a first end 111 that is adjacent to the second end 109 of the shoulder region 107 and a second end 112, and a flange region 113 which, at least in parts of the flange region 113, is formed in the shape of a plate, the flange region 113 having a first end 114 that is adjacent to the second end 112 of the body region 110 and a second end 115 that corresponds to the top end 102 of the glass syringe barrel 100, wherein the flange region 113 has an outer diameter d₂>d₁. As also shown in FIG. 2, the length of the body region 110 is l₁ and the length of the upper most end on top of the flange region 113 determined at position P₂ to the bottom end (i. e. the lower end of the conically shaped region) is l₂.

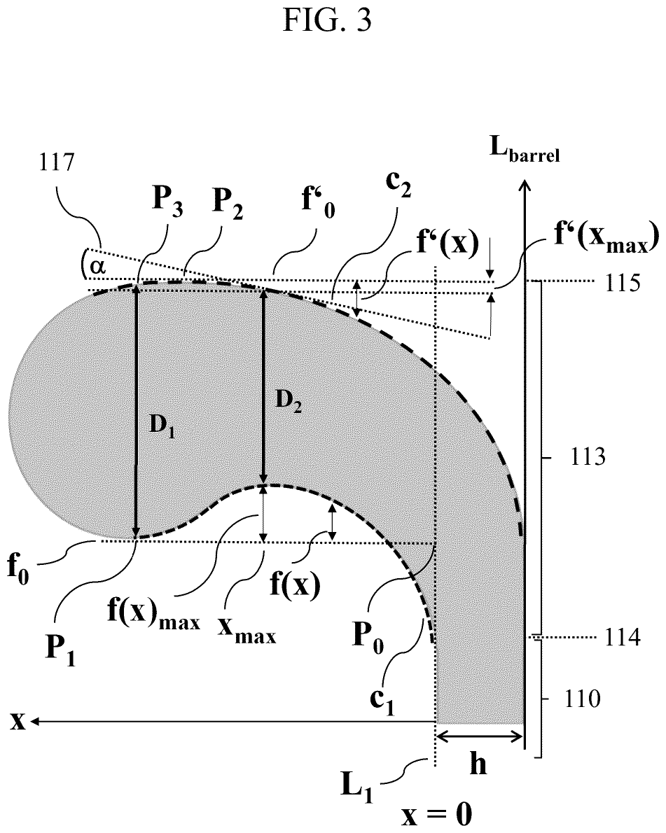

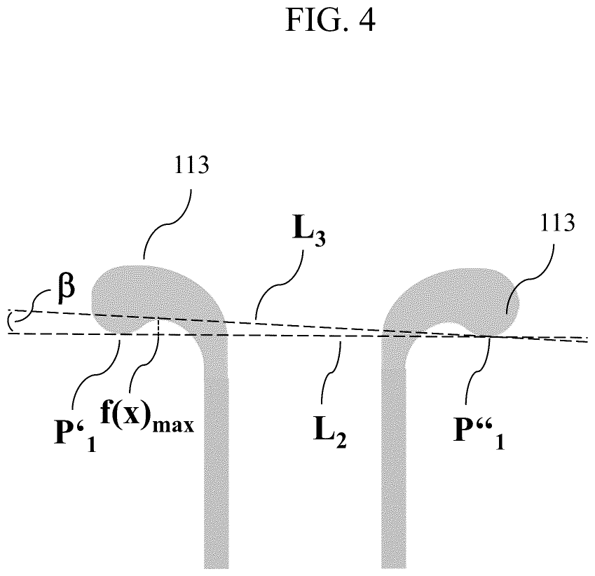

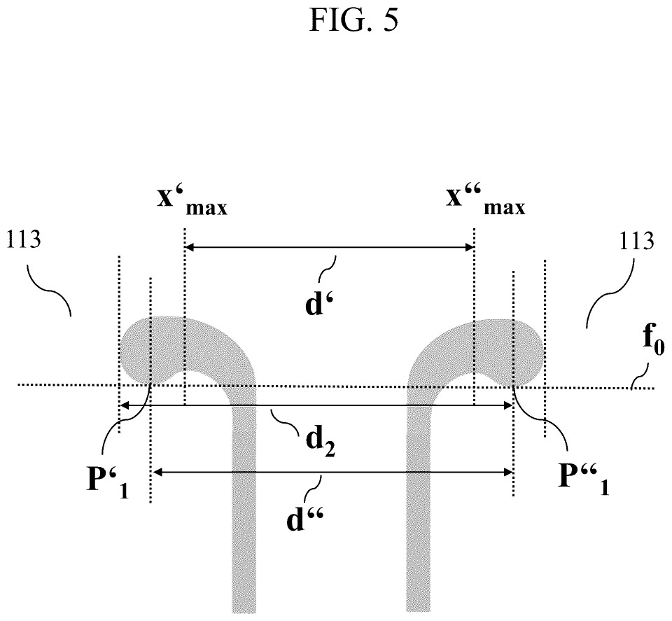

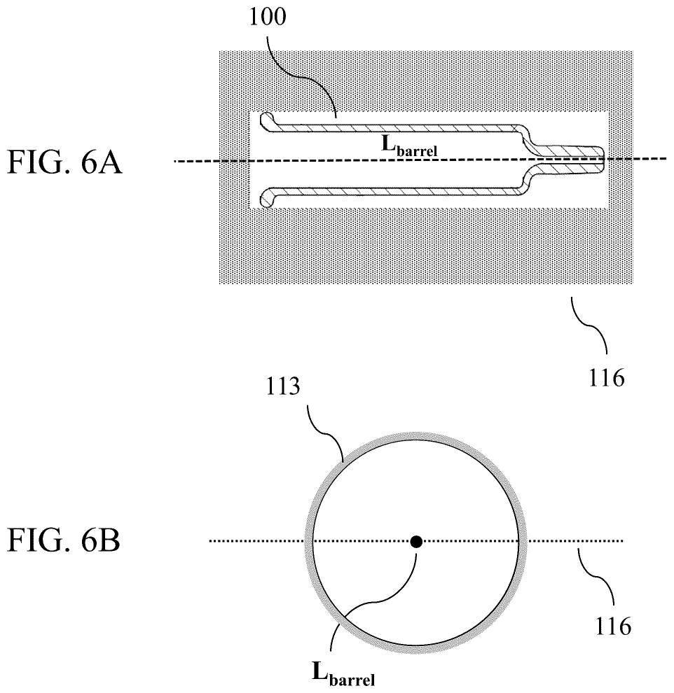

FIG. 3 shows a cross-sectional enlarged view of a part of the flange region 113 in a glass syringe barrel 100 according to the present invention and the determination of the outer contour c1 and c2 by means of functions f(x) and f′(x). FIG. 3 just shows the left part of the flange region in a cross-section of the glass syringe barrel 100 that is located in a plane 116 being centrically located in the glass syringe barrel 100 and comprising the longitudinal axis L_{barrel} of the glass syringe barrel (see FIGS. 6A and 6B). The corresponding part on the right (which is shown in FIGS. 4 and 5) is not shown in FIG. 3. As can be seen in FIG. 3, flange region 113 has an outer diameter d₂>d₁ and is characterized by an outer contour c1 at its first end 114 (dotted lined on the top surface of the flange region 113) and an outer contour c2 at its second end 115 (dashed line on the bottom of the flange region 113). fₒ and f′ₒ are straight lines that run vertically to longitudinal axis L_{barrel}, wherein fₒ touches the outer contour c1 at its deepest point P₁ and f′ₒ touches the outer contour c2 at its highest point P₂. As can also be seen in FIG. 3, f(x) defines the absolute value of the vertical distance between any point of the outer contour c1 and straight line fₒ at a position x (with f(x)=0 at point P₁), wherein x is the horizontal distance between any given point on straight line fₒ and point P₀ at which straight line fₒ crosses a line L₁ that runs parallel to longitudinal axis L_{barrel} and that touches the outer surface of the body region 110. The maximum value for the term f(x) in the range from x=P₀ to x=P₁ is f(x)ₘₐₓ determined at position xₘₐₓ. f′(x) defines the absolute value of the vertical distance between any point of the outer contour c2 and straight line f′ₒ at a position x (with f′(x)=0 at point P₂), The glass syringe barrel according to the present invention is characterized in that the flange region 113 has a shape such that condition f′(xₘₐₓ)/f(x)ₘₐₓ>1 is fulfilled. As can also be seen in FIG. 3, D1 is the distance between point P₁ on the outer contour c1 and a point P₃ on outer contour c2, point P₃ being located vertically above point P₁(=thickness of the flange region at its deepest point). According to a preferred embodiment of the glass syringe barrel according to the present invention condition D₁/D₂<1.5 is fulfilled.

FIG. 4 shows in a further a cross-sectional enlarged view of the shape of a flange region 113 in a glass syringe barrel 100 according to the present invention and the determination of R. As shown in FIG. 4, L₂ is a straight line at touches the deepest point P′₁ of the outer contour c1 on the left side of the flange region 113 (that is also shown in FIG. 3) and the deepest point P″₁ of the outer contour c1 on the right side of the flange region 113 (that is not shown in FIG. 3). L₃ is a straight line at touches the outer contour c1 on the left side of the flange region 113 at a position x at which f(x) reaches the maximum value f(x)ₘₐₓ and the deepest point P″₁ of the outer contour c1 on the right side of the flange region 113. According to a preferred embodiment of the glass syringe barrel according to the present invention condition straight lines L₂ and L₃ enclose an angle β of less than 2.5 degree.

FIG. 5 shows in a further a cross-sectional enlarged view of the shape of a flange region 113 in a glass syringe barrel 100 according to the present invention and the determination of d′ and d″. As shown in FIG. 5, d′ is the distance between point x′ₘₐₓ on the left side of the flange region 113 and point x″ₘₐₓ on the right side of the flange region 113 on straight line fₒ, x′ₘₐₓ and x″ₘₐₓ corresponding to the points at which function f(x) reaches its maximum value on the left side and the right side of the flange region 113, respectively. According to a preferred embodiment of the glass syringe barrel according to the present invention d′ is in the range from 9 to 14 mm. As can also be seen in FIG. 5, d″ is the distance between point P′₁ on the left side of the flange region 113 and point P″₁ on the right side of the flange region 113 on straight line fₒ. According to a further preferred embodiment of the glass syringe barrel according to the present invention wherein d″ is preferably in the range from 8 to 20 mm. According to a further preferred embodiment of the glass syringe barrel according to the present invention, d″/d₂ is in the range from 0.8 to 0.95.

FIGS. 6A and 6B show in a side view and in a top view the localization of plane 116 in the glass syringe barrel 100 that is used to determine the outer contour c1 and c2 in the flange region 113. Plane 116 corresponds to the plane that is centrically located in the glass syringe barrel 100 and that comprises the longitudinal axis L_{barrel} of the glass syringe barrel 100.

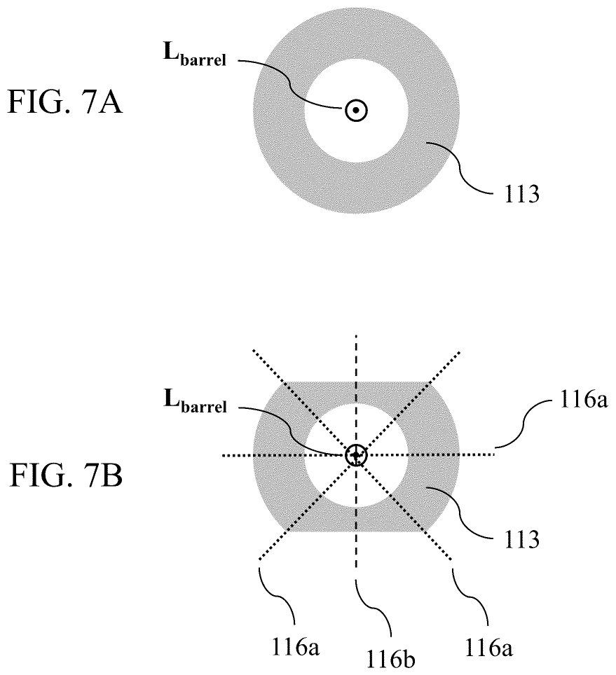

FIGS. 7A and 7B show a top-view two different shapes of the flange region 113. The flange region 113 shown in FIG. A as a (full) circular shape, whereas the flange region 113 shown in FIG. 7B has been derived from a flange region 113 by cutting off pieces of the flange region 113 on two opposite sides. In case of such “cut-flange”, plane 116 that is used to determine the outer contour c1 and c2 in the flange region 113 as shown in FIGS. 6A and 6B is located in a plane being centrically located in the glass syringe barrel 100 at a position at which the outer contour of the flange region 113 is circular shaped. Planes 116 that fulfil this requirement are the dotted lines 116a, whereas a plane that does not fulfil this requirement is dashed line 116b.

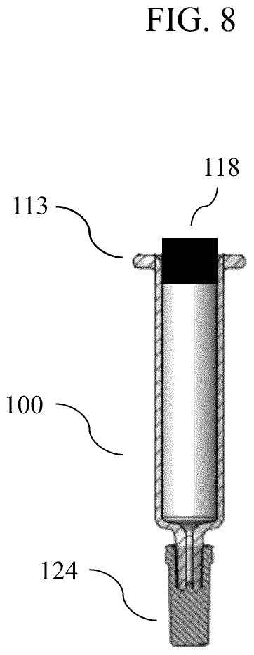

FIG. 8 shows a glass syringe barrel 100 according to the present invention with a tip cap 124 attached to the conically shaped side at the lower end and with a plug 118 that is inserted into the open end of the glass syringe barrel 100 at the upper end at which the flange region 113 is located.

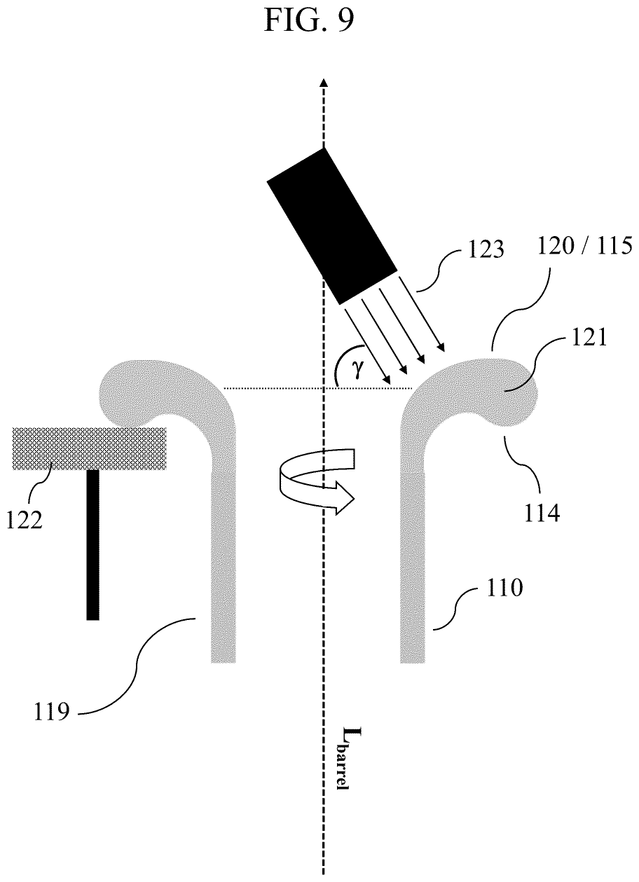

FIG. 9 illustrates step III) of process 1 according to the invention for the preparation of a glass syringe barrel 100. In the process for the preparation of a glass syringe barrel 100 according to the present invention in a first process step a glass syringe barrel precursor 119 is provided having a longitudinal axis L_{barrel}, the glass syringe barrel precursor 119 comprising a body region 110 with a bottom end and a top end, the body region 100 preferably having an outer diameter d₁. In a particular embodiment of the process according to the present invention, the bottom end is already shaped in the form of a conus to obtain the cone region 104 as shown in FIG. 2. In a further process step the top end 120 of the body region 110, while rotating the glass syringe barrel precursor 119 around its longitudinal axis L_{barrel}, is heated to prepare a ring of molten glass 121 at the top end, wherein heating is preferably accomplished by means of flames from a gas burner that are directed onto the top surface 115 of the glass syringe barrel precursor 119 to obtain a molten ring of glass 121. A flange region 113 is formed by pushing the glass mass 121 out beyond the edge of the body region 110 at the top end of the glass syringe barrel precursor 119. In process step III) of the process according to the present invention (shown in FIG. 9), the thus obtained flange region 113 is finally shaped to obtain a bottom side 114 having an outer contour c1 and a top side 115 having an outer contour c2. As shown in FIG. 9, the final shape on the bottom side 114 of the flange region 113 is prepared by bringing the mass of molten glass 121 into contact with a shaping tool 122 in such a way that the shaping tool 122 somehow lifts the mass of glass 121 upwards, thereby reducing the deepest indentation on the lower side of the flange region 113 (f(x)ₘₐₓ). At the same time, the top side 115 of the flange region 113 is cooled by means of an air stream 123 (applied in an angle of approximately 50°) that primarily serves to cool down the surface of the flange region 113 on the top side 115, thereby stabilizing the shape of the flange region 113 on that side.

### LIST OF REFERENCE NUMERALS

100 glass syringe barrel according to the invention
101 bottom end
102 top end
103 plunger stopper
104 cone region
105 first end of the cone region 104 (=bottom end 101)
106 second end of the cone region 104 (=first end 108 of the shoulder region 107)
107 shoulder region
108 first end of the shoulder region 107 (=second end 106 of the cone region 104)
109 second end of the shoulder region 107 (=first end 111 of the body region 110)
110 body region
111 first end of the body region 110 (=second end 109 of the shoulder region 107)
112 second end of the body region 110 (=first end 114 of the flange region 113)
113 flange region
114 first end (bottom side) of the flange region 113 (=the second end 112 of the body region 110)
115 second end (top side) of the flange region 113 (=top end 102)
116 cross-sectional plane in the middle of the glass syringe barrel 100
117 a tangent that touches outer contour c2 at position xₘₐₓ
118 plug
119 glass syringe barrel precursor
120 top end of glass syringe barrel precursor 119
121 ring of molten glass
122 shaping tool
123 gas stream
124 tip cap

What is claimed is:

1. A glass syringe barrel, comprising:
a bottom end through which a liquid can be ejected;
a top end into which a plunger stopper can be pushed; and
a longitudinal axis defined through the bottom and top ends;
wherein the glass syringe barrel, in a direction from the bottom end to the top end, includes:
a cone region having a first end that corresponds to the bottom end and a second end;
a shoulder region having a third end adjacent to the second end and a fourth end;
a body region with a first outer diameter (d₁) having a fifth end that is adjacent to the fourth end and a sixth end; and
a flange region having, at least in parts, a circular shape, the flange region having a seventh end that is adjacent to the sixth end and an eighth end that corresponds to the top end, wherein the flange region has a second outer diameter (d₂), the second outer diameter (d₂) being larger than the first outer diameter (d₁),
wherein the flange region has a first outer contour at the seventh end and a second outer contour at the eighth end;
wherein for any cut surface that includes the longitudinal axis and is obtainable by cutting the glass syringe barrel in a plane at which a shape of the flange region is circular:
fₒ is a straight line that runs perpendicular to the longitudinal axis and touches the first outer contour at a deepest point (P₁),
f′ₒ is a straight line that runs perpendicular to the longitudinal axis and touches the second outer contour at a highest point (P₂),
f(x) defines an absolute value of a vertical distance between any point of the first outer contour and the straight line fₒ at a position x, with f(x)=0 at point P₁, wherein x is a horizontal distance between any given point on the straight line fₒ and point P₀ at which the straight line fₒ crosses a line L₁ that runs parallel to the longitudinal axis and that touches an outer surface of the body region, and a maximum value for the term f(x) in a range from x=P₀ to x=P₁ is f(x)ₘₐₓ determined at position xₘₐₓ;
wherein f′(x) defines an absolute value of the vertical distance between any point of the second outer contour and the straight line f′ₒ at a position x, with f′(x)=0 at point P₂, and
wherein the following condition is fulfilled: f′(xₘₐₓ)/f(x)ₘₐₓ>1.

2. The glass syringe barrel of claim 1, wherein f′(xₘₐₓ) is in a range from 0.15 to 0.7 mm.

3. The glass syringe barrel of claim 1, wherein f(x)ₘₐₓ is in a range from 0.01 to 0.65 mm.

4. The glass syringe barrel of claim 1, wherein D1 is a distance between the point P₁ on the first outer contour and a point P₃ on the second outer contour, wherein the point P₃ is located vertically above point P₁ and D₂ is a distance between a point of the first outer contour and the outer contour at position xₘₐₓ and wherein the following condition is fulfilled: D₁/D₂<1.6.

5. The glass syringe barrel of claim 1, wherein d′ is a distance between point x′ₘₐₓ on a left side of the flange region and point x″ₘₐₓ on a right side of the flange region on the straight line fₒ, x′ₘₐₓ and x″ₘₐₓ corresponding to points at which the function f(x) reaches a maximum value on the left side and the right side, wherein d′ is in a range from 12.5 to 15 mm.

6. The glass syringe barrel of claim 1, wherein d″ is a distance between point P′₁ on a left side of the flange region and point P″₁ on a right side of the flange region on the straight line f₀, wherein d″/d₂ is in a range from 0.8 to 0.95.

7. The glass syringe barrel of claim 6, wherein d″ is in a range from 8 to 20 mm and the second outer diameter (d₂) is in a range from 12 to 22 mm.

8. The glass syringe barrel of claim 1, wherein the first outer diameter (d₁) is in a range from 6 to 20 mm.

9. The glass syringe barrel of claim 1, wherein a tangent that touches the second outer contour at position xₘₐₓ includes an angle α with the straight line f′ₒ and wherein the angle α is in a range from 10 to 28 degrees.

10. The glass syringe barrel of claim 1, wherein:
L₂ is a straight line that touches the deepest point P′₁ of the first outer contour on the left side of the flange region and the deepest point P″1 of the first outer contour on the right side of the flange region;
L₃ is a straight line that touches the first outer contour on the left side of the flange region at a position x at which f(x) reaches the maximum value f(x)ₘₐₓ and the deepest point P″₁ of the first outer contour on the right side of the flange region; and
the straight lines L₂ and L₃ enclose an angle β of less than 3 degree.

11. The glass syringe barrel of claim 1, wherein d₂−d₁ is in a range from 5 to 8 mm.

12. The glass syringe barrel of claim 1, wherein the flange region, in a plane that is perpendicular to the longitudinal axis, has the shape of a circle or the shape of a circle where two opposite sections of the circle have been removed.

13. A nest box comprising:
a plurality of the glass syringe barrels of claim 1, wherein at least one of the following conditions is fulfilled: a relative standard deviation of a flange thickness is less than 6%; and a relative standard deviation of a length of the glass syringe barrels is less than 12%.

14. The nest box of claim 13, wherein both conditions are fulfilled.

15. A syringe comprising:
the glass syringe barrel of claim 1; and
a plunger stopper pushed into the top end.

16. The syringe of claim 15, further comprising a pharmaceutical composition in the glass syringe barrel.

17. A process for the preparation of a glass syringe barrel, comprising:
providing a glass syringe barrel precursor having a longitudinal axis, the glass syringe barrel precursor comprising a body region with a bottom end and a top end;
heating the top end, while rotating the glass syringe barrel precursor around the longitudinal axis, to prepare a ring of molten glass at the top end and forming a flange region by pushing the ring of molten glass out beyond an edge of the body region at the top end; and
shaping, while the glass syringe barrel precursor is rotating around the longitudinal axis, the flange region to obtain a bottom side with a first outer contour and a top side with a second outer contour, wherein the bottom side is shaped by contact with a shaping tool.
